(19) United States
(12) Patent Application Publication
(10) Pub. No.: US 2007/0213776 A1
(43) Pub. Date: Sep. 13, 2007

(54) High-Voltage Module for An External Defibrillator

(75) Inventor: Gregory D. Brink, Bainbridge Island, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., EINDHOVEN (NL)

(21) Appl. No.: 11/575,770

(22) PCT Filed: Sep. 12, 2005
(86) PCT No.: PCT/IB05/52983

§ 371 Date: Mar. 22, 2007

Related U.S. Application Data
(60) Provisional application No. 60/614,452, filed on Sep. 29, 2004.
(60) Provisional application No. 60/651,432, filed on Feb. 8, 2005.

(51) Int. Cl. A61N 1/39

(52) U.S. Cl. 607/7; 607/5

(57) ABSTRACT

A defibrillator includes a module having a portion of the high-voltage components of the defibrillator attached to a substrate and encased in a dielectric material. In one embodiment the defibrillating shock is delivered by a high voltage H-bridge circuit which utilizes four controllably switched semiconductor devices such as IGBTs.

The present invention relates generally to external defibrillators, and more particularly, relates to defibrillators having compact and modular designs for high-voltage components of the defibrillator.

Sudden cardiac arrest (SCA) most often occurs without warning, striking people with no history of heart problems. It is estimated that more than 1000 people per day are victims of sudden cardiac arrest in the United States alone. SCA results when the electrical component of the heart no longer functions properly causing an arrhythmia. One such arrhythmia, ventricular fibrillation (VF), is caused by abnormal and very fast electrical activity in the heart. As a result, the heart fails to adequately pump blood through the body. VF may be treated by applying an electric shock to a patient's heart through the use of a defibrillator.

Defibrillators include manual defibrillators, automatic or semi-automatic external defibrillators (AEDs), defibrillator/monitor combinations and advisory defibrillators. The shock delivered by a defibrillator clears the heart of abnormal electrical activity (in a process called “defibrillation”) by producing a momentary asystole and providing an opportunity for the heart's natural pacemaker areas to restore a normal cardiac rhythm. Currently available external defibrillators provide either a monophasic or biphasic electrical pulse to a patient through electrodes applied to the chest. Monophasic defibrillators deliver an electrical pulse of current in one direction, whereas biphasic defibrillators deliver an electrical pulse of current first in one direction and then in the opposite direction. When delivered external to the patient, these electrical pulses are high energy pulses, typically in the range of 50 Joules for pediatric patients to 200 Joules for adults.

External defibrillators are typically located and used in hospital emergency rooms, public facilities, and emergency medical vehicles. Of the wide variety of external defibrillators currently available, automatic and semi-automatic external defibrillators (AEDs) are becoming increasingly popular because they can be used by relatively inexperienced personnel. Such defibrillators can also be especially lightweight, compact, and portable, enabling their immediate use even when experienced medical personnel have not arrived to attend to a patient. In order to do this, the medical expertise of the medical professional must be replaced with precise information processors which can be programmed to analyze an ECG waveform and reliably determine if and when a defibrillating shock is to be applied to the patient. Additionally it is desirable that AEDs be as lightweight and compact as possible for easy portability.

These demands mean that low voltage processors and integrated circuitry must share an AED package with the high voltage components of the shock delivery circuitry. To prevent interference or damage to the low voltage circuitry care must be taken in locating components in the package so that the low voltage components are sufficiently isolated and separated from the high voltage components. Generally this has been accomplished by locating the high voltage components on their own area of the unit's printed circuit board and by following component layout and design rules which adequately separate the high voltage components from the low voltage components and conductors. Unfortunately abiding by these design rules tends to make an AED bulkier than is otherwise desirable. Accordingly it is desirable to be able to package the components of an AED as compactly as possible and, if possible, even more compactly than the design rules would dictate, but without exposing the low voltage components and conductors to the hazards of arcing or discharge from high voltage conductors and components of the unit.

In accordance with the principles of the present invention high voltage components of an AED unit are packaged together and separated from other components by a non-air dielectric which insulates the high voltage components and prevents damage to nearby low voltage components and conductors. The non-air dielectric enables the high voltage components to be packaged more densely than the separation design rules would otherwise allow, aided by the low duty cycle of their use which limits the need for heat dissipation.

In accordance with a further aspect of the present invention the high voltage bridge circuit used to deliver pulses to the patient of a desired period and polarity is formed primarily or entirely of IGBT devices. The IGBT devices enable the switching elements of the bridge circuit to be controllably turned off, enabling the circuit to be used for related lower voltage applications such as pacing.

In the drawings:

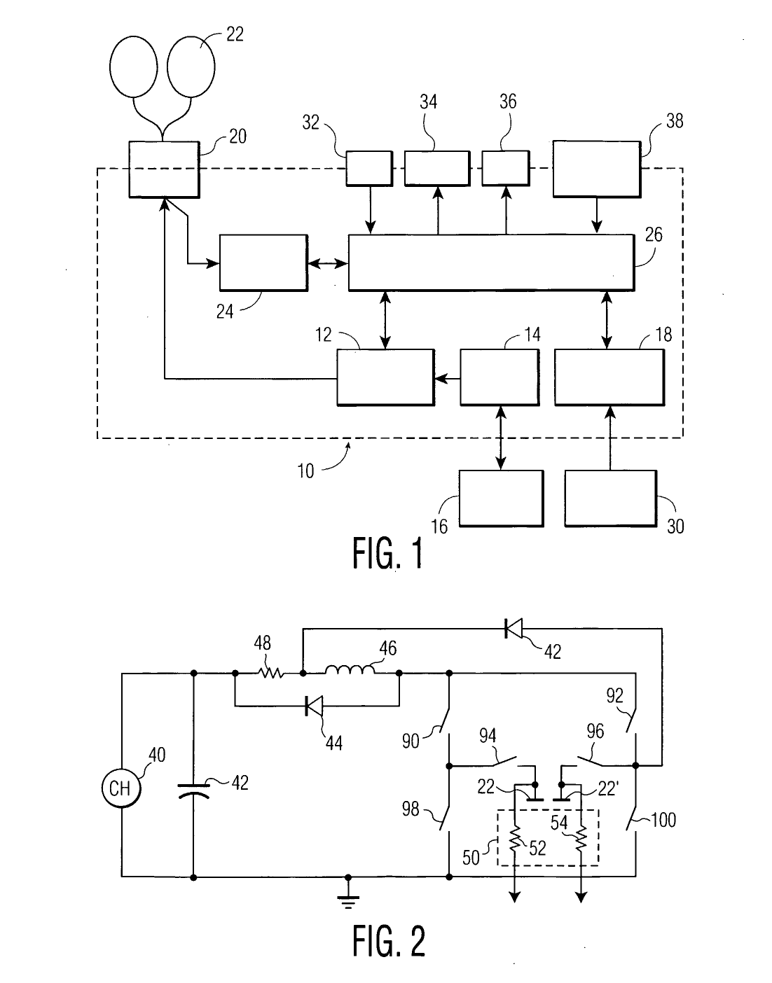

FIG. 1 is a functional block diagram of an external defibrillator according to an embodiment of the present invention.

FIG. 2 is a functional block diagram of a high-voltage delivery circuit included in the defibrillator of FIG. 1.

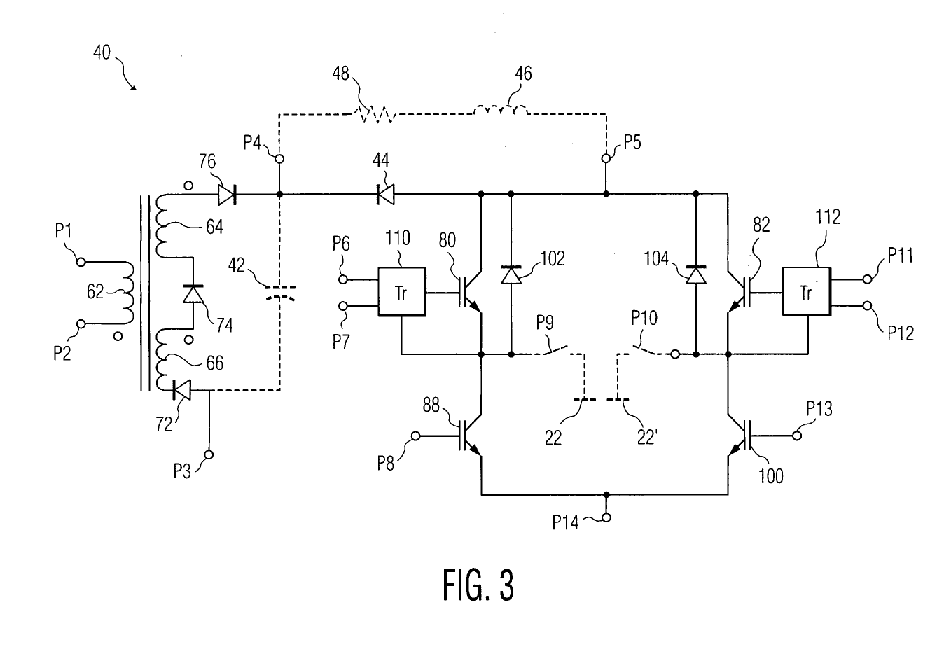

FIG. 3 is a schematic diagram showing high voltage components which are modularly packaged in accordance with the principles of the present invention.

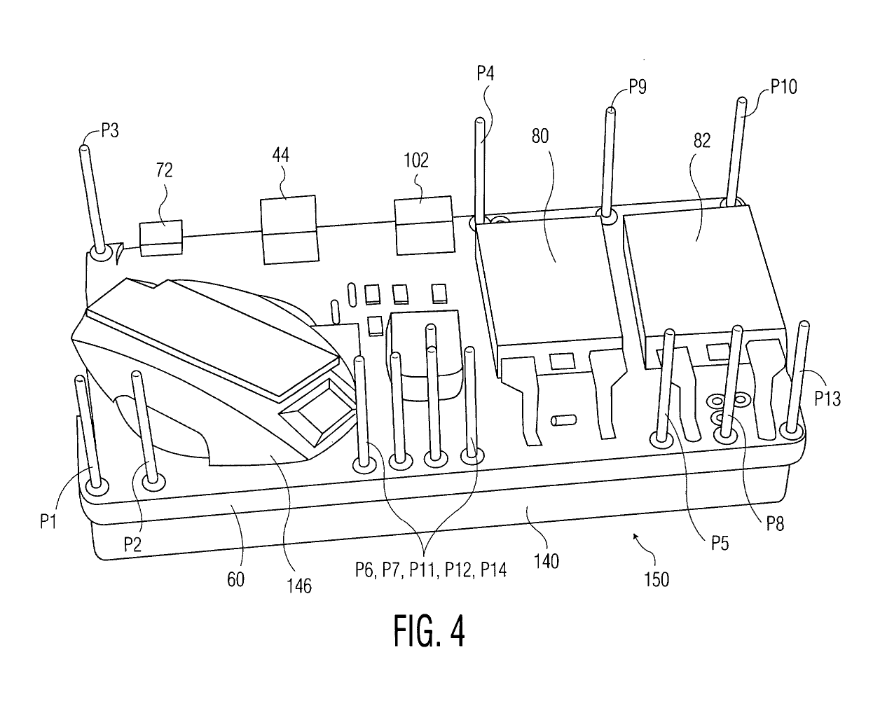

FIG. 4 is a perspective view of one side of a high-voltage module according to an embodiment of the present invention.

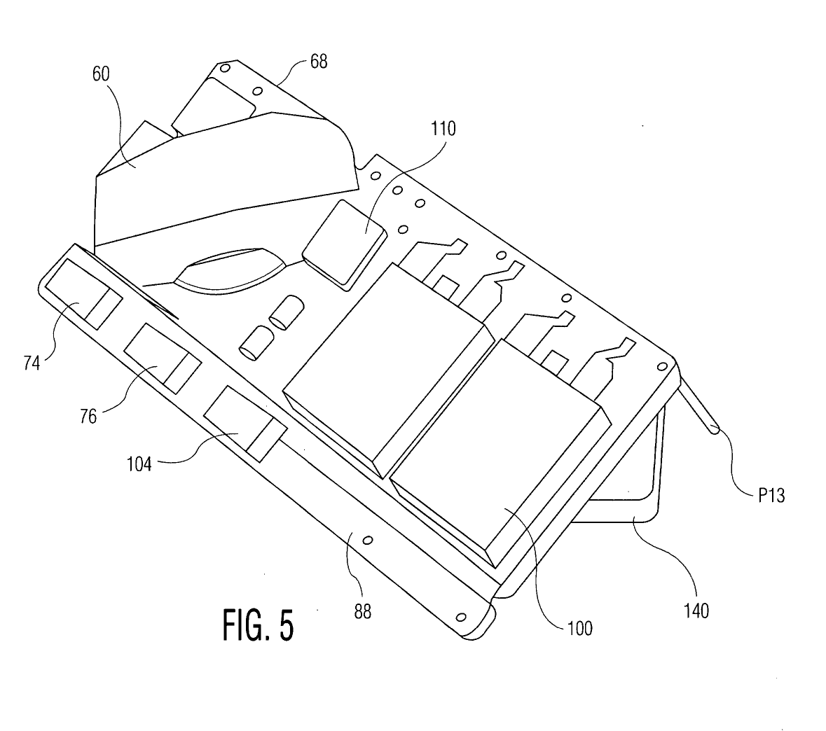

FIG. 5 is a perspective view of the second side of the high-voltage module of FIG. 4.

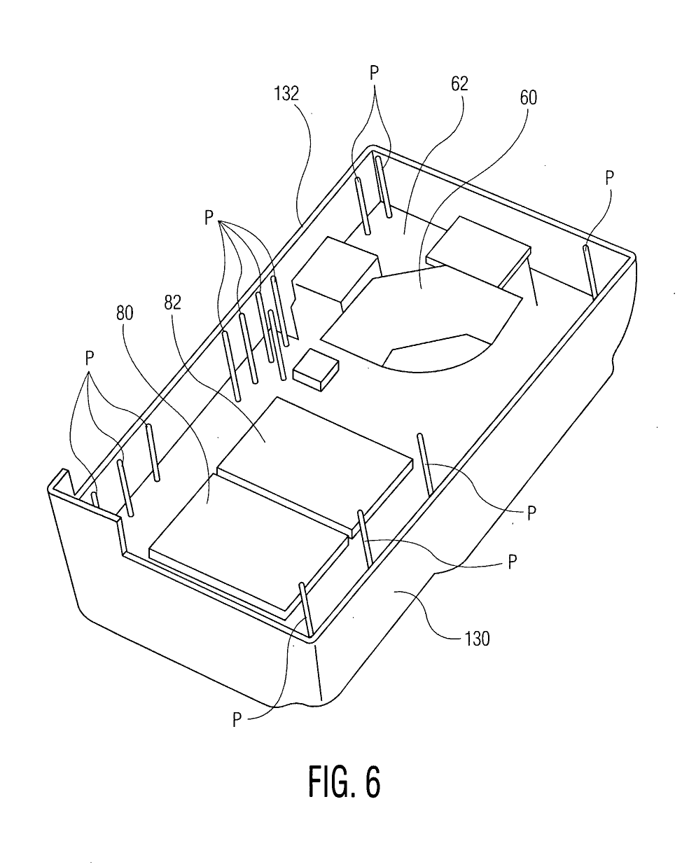

FIG. 6 is a perspective view of the high-voltage module of FIG. 5 positioned in a potting cup.

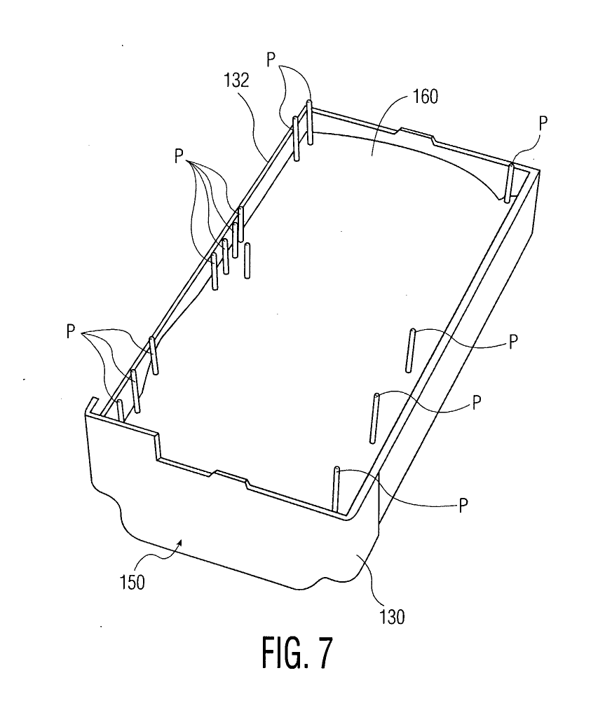

FIG. 7 is a perspective view of the high-voltage module of FIG. 5 encased by a potting material.

Embodiments of the present invention are directed to a defibrillator that includes a compact high-voltage module having high-voltage electronics of the defibrillator placed in proximity to one another and encased in a dielectric material. In the following description well-known circuits have not been shown in detail in order to avoid unnecessarily obscuring the description of the various embodiments of the invention. Also not presented in any great detail are those well-known control signals and signal timing protocols associated with the internal operation of defibrillators.

FIG. 1 is a functional block diagram of a defibrillator or AED 10 according to an embodiment of the present invention. The AED 10 includes a delivery circuit 12 that is capable of delivering high or low voltage depending upon the application. The AED 10 further includes a power supply 14 which is powered by an energy source such as a removable battery 16 which provides power to components of the AED 10, including the high-voltage delivery circuit 12. A microcontroller or processor 18 controls the operation of the various components of the AED 10. The high-voltage delivery circuit 12 delivers a pulse of electrical energy to a patient via an electrode connector or interface 20 and patient electrodes 22.

An electrocardiogram (ECG) circuit 24 acquires and preconditions the patient's ECG signals acquired through the electrodes 22 and sends the signals to the processor 18 via a system gate array 26. The system gate array 26 is a custom application-specific integrated circuit (ASIC) integrating many of the defibrillator functions (including user interface control and many of the internal functions) and interfacing the processor 18 with other components of the AED 10. Providing the separate system gate array or ASIC 26 allows the processor 18 to focus on other tasks. The functionality of the ASIC 26 can be included within the operations performed by the processor 18 as well, or can be replaced by discrete logic circuit components or a separately dedicated processor.

The AED 10 also includes a memory device 30 (such as a removable Personal Computer Memory Card International Association (PCMCIA) card, Secure Digital card or flash memory), and user interface components such as a microphone 32, an audio speaker 34, an LCD display panel 36, and a set of push-button controls 38. Those skilled in the art will understand that a number of other components may be included within the AED 10 (e.g., a system monitor and associated status indicators), but are not shown in order to avoid unnecessarily obscuring the description of embodiments of the present invention.

The high voltage components of the AED of FIG. 1 are found in the power supply 14 which develops the high voltages needed for defibrillation and in the delivery circuit 11 which delivers the high voltage to the electrodes 22 and ultimately the patient. FIG. 2 is a schematic and block diagram illustration of the high voltage section of an AED constructed in accordance with the present invention. A high voltage power supply or charger 40 operates to charge the energy storage capacitor 42 for delivery of a defibrillating pulse. The high energy pulse is applied by way of a resistor 48 which serves to limit the current on discharge of the capacitor. During normal pulse delivery switches 94 and 96 are closed to connect the energy delivery circuit 12 to the electrodes 22 which are applied to the patient. Switches 94 and 96 are safety switches which are opened when the capacitor 42 is charged while the patient's ECG waveform is being monitored to prevent accidental discharge of high voltage to the patient and ECG circuits during charging. Four switches 90, 92, 98 and 100 are used in conjunction with switches 94 and 96 in an “H-bridge” configuration. When a biphasic pulse is to be delivered to the patient, switches 90 and 100 are closed and switches 92 and 98 are left opened. The high voltage energy will then flow through the closed switches and from the left electrode 22 and through the patient to the right electrode 22′. After the first phase of the biphasic pulse has been delivered the second phase of the pulse is delivered by opening switches 90 and 100 and closing switches 92 and 98. The high voltage current will then flow through the patient in the opposite direction, from electrode 22′, through the patient, and through electrode 22. Thus, the H-bridge delivery circuit is a means by which a biphasic pulse can be delivered to the patient.

Also connected to the electrodes 22 is an ECG front end circuit 50 by which the electrodes 22 are used to detect the patient's ECG waveform when defibrillating pulses are not being delivered. When the patient's ECG waveform is being detected and analyzed the safety switches 94 and 96 are opened and the patient's ECG signal is applied to the input of one or more ECG input amplifiers across the impedances 52 and 54.

The delivery circuit 12 in the illustrated embodiment also includes a series inductor 46 which, together with the resistor 48, serves to limit the rate of current rise of an applied high voltage pulse. A diode 44 clamps the inductance of the inductor 46 any time that current in the delivery circuit is turned off, such as at the end of the first phase of the pulse. A diode 42 clamps the inductance of the electrode wires to the patient at the end of the first phase of the pulse.

In one embodiment the switches 90, 92, 98 and 100 are not mechanical switches but are solid-state switching devices. In this embodiment switches 90, 92, and 98 are SCRs (silicon-controlled rectifiers) and switch 100 is an IGBT (insulated gate bipolar transistor). With safety switches 94 and 96 closed, a biphasic pulse is delivered to the patient by first switching SCR 90 and IGBT 100 to the conductive state. There is an initial rise in current after which the positive pulse undergoes a controlled decay. After a predetermined time for the positive pulse the IGBT 100 is opened and the voltage and current of the positive pulse drop to zero. SCR 90 then returns to a blocking state. Shortly thereafter SCRs 92 and 98 are switched to the conductive state and there is a rapid rise of the negative pulse, followed by a controlled decay of the pulse. After a predetermined period for the negative pulse SCR 90 is made conductive, bringing the current and voltage applied to the patient to zero again. When the flow of current stops the SCRs turn off and return to the blocking state. The H-bridge is then in its initial state and ready for the next pulse sequence.

In accordance with the principles of the present invention high voltage components of the high voltage capacitor charging circuit, the high voltage delivery circuit and the H-bridge circuit are assembled in a high voltage module. In a constructed embodiment the module includes a multi-layer circuit board on which the H-bridge semiconductor switches and diodes, isolation transformers and control circuits for the semiconductor switches, and the transformer and diodes of the high voltage capacitor charging circuit are mounted. The constructed embodiment utilizes standard discrete surface mount semiconductor packages, control circuits and isolation transformers. The constructed module also integrates a planar transformer for the capacitor charger which utilizes etched conductors on the circuit board for the transformer windings. By encapsulating the module in a dielectric such as an epoxy, urethane, silicone, acrylic, or polyester resin, the creepage and clearance distances needed to prevent arcing can be reduced, enabling a more compact package. By combining a plurality of high voltage components into a single module only one potting cup and encapsulation process is required, which reduces the cost of system manufacture.

Referring to FIG. 3 the components of a high voltage module constructed in accordance with the principles of the present invention are shown in solid lines. The module is mounted on the AED system circuit board (not shown), where connections are made to other components shown in dashed lines. Fourteen pins, labeled P1 through P14, are used to connect the circuitry of the high voltage module to other conductors and components of the AED. At the left side of the drawing is the charger circuit 40, including a transformer having a primary coil 62 and secondary coils 64 and 66. The secondary windings are separated by diodes 72, 74, and 76 which provides symmetrical design in which stray capacitances are balanced. In a constructed embodiment the charger transformer is formed as a planar transformer in which the coils 62, 64, and 66 are formed by opposing areas of etched copper of the module printed circuit board around which ferrite core halves forming the transformer core are clamped. The high voltage capacitor 42 is connected to pins P3 and P4 of the module. Pins P4 and P5 are connected to the external current limiting resistor 48 and inductor 46. The clamping diode 44 is located on the module and connected between pins P4 and P5.

In accordance with a further aspect of the present invention, in this embodiment the switching devices 90, 92, 98 and 100 are provided by four semiconductor IGBT devices 80, 82, 88, and 100. High voltage IGBT devices are used, preferably with a voltage rating in excess of 2000 volts. The use of IGBTs for all of the switching devices provides better control of energy delivery over a full range of 2-200 Joules. The IGBTs will remain conductive at very low current levels and can be controllably turned off, as opposed to SCRs which remain conductive so long as a sufficient supply of current is available. Once turned on, SCRs will remain conductive as long as at least the minimum hold current is maintained. The SCRs will not turn off until the current supply is reduced below the minimum hold current for the devices. The use of IGBTs for the switching devices of the H-bridge enables an embodiment of the present invention to be used for lower voltage applications such as pacing in addition to defibrillation. The IGBTs are also not as sensitive to rapid current and voltage rises as are SCRs. The collectors of the upper pair of IGBTs 80 and 82 are coupled to the pin P5 to receive the high voltage and current from the high voltage capacitor 42. The emitters of the IGBTs 80 and 82 are coupled to pins P9 and P10 which connect to the electrodes 22. Shunt diodes 102 and 104 are coupled across the collector-emitter paths of the IGBTs 80 and 82. The IGBTs are controlled by driver circuits including gate drive transformers coupled to the gates of the IGBTs 80 and 82. The two lower IGBTs 88 and 100 of the H-bridge circuit are coupled between the upper IGBTs and pin P14, which is coupled to reference potential.

FIG. 4 illustrates the high voltage circuit elements of FIG. 3 assembled in a unitary high voltage module 150. (A US one cent piece is shown behind the module to enable the viewer to gauge the size of the module.) The components are mounted on a substrate which in this embodiment is a multi-layer printed circuit board 140. The connecting pins P1-P14 are seen to extend upward from the board 140 in this picture. In this embodiment the components are mounted on both sides of the printed circuit board in a symmetrical arrangement. The planar transformer 60 is located on the left side of the board 140. A thin disk of white plastic is partially visible which is an insulator for the transformer coil etched on this side of the printed circuit board. The white plastic disk is mostly obscured in this view by the ferrite core half clamped over the disk which forms a portion of the core of the planar transformer. Behind pins P6, P7, P11, P12, P14 is one of the isolation transformers of an IGBT trigger circuit 112 together with surface mounted components of the trigger circuit. At the rear of the board are three of the diodes 72, 44 and 102. On the right side of the board are two of the IGBT devices 80 and 82.

FIG. 5 shows the reverse side of the printed circuit board 140. The other half of the planar transformer 60 is seen at the top of the illustration, including the other portion of the ferrite core 68. The other three diodes of the module, 74, 76, 104 are mounted opposite the first three diodes on the other side of the board. The isolation transformer of trigger circuit 110 is seen in the center of the board, opposite the isolation transformer on the other side of the board. IGBT devices 88 and 100 are mounted opposite the IGBT devices on the other side of the board.

In FIG. 6 the printed circuit board 140 with its mounted components is shown in a plastic potting cup 130. The fourteen pins, all labeled P in this illustration, extend upward in this view above the edge 132 of the potting cup. The potting cup 130 is then filled with a potting compound 160 which completely encapsulates the board and high voltage components as shown in FIG. 7. The potting compound thereby provides an insulating dielectric around and between all of the high voltage components. The potted board and components can be removed from the potting cup and used in that con figuration. In the constructed embodiment the potted board and components is left in the potting cup and the entire high voltage module 150 including the potting cup 130 is mounted on the AED system printed circuit board. The module is compact and takes up less space on the system printed circuit board than would the individually mounted components, and the potting cup and dielectric potting compound provide an insulation layer which prevents arcing between the high voltage components and nearby low voltage nodes, conductors, and components.

Other embodiments will readily occur to those skilled in the art. For example, instead of arranging the switching devices in a full H-bridge circuit, only a half-bridge circuit consisting of two switching devices on one side of the bridge and two capacitors on the other side of the bridge may be used.

1. An external defibrillator comprising:
a pair of patient electrodes for delivering energy to a patient;
a processor, responsive to signals received by the electrodes, which processes an ECG signal;
an energy storage device for storing high voltage electrical energy;
a charging circuit for charging the energy storage device; and
a bridge circuit for delivering energy from the energy storage device to the electrodes, the bridge circuit having legs comprising a plurality of high voltage semiconductor devices coupled to the electrodes, a plurality of the bridge circuit legs including high voltage semiconductor devices which can be controllably switched to a nonconducting state.

2. The external defibrillator of claim 1 wherein bridge circuit comprises an H-bridge including
first and second semiconductor devices each having a high voltage conductive path coupled between the energy storage device and an electrode; and
third and fourth semiconductor devices each having a high voltage conductive path coupled between an electrode and a reference potential.

3. The external defibrillator of claim 1 wherein the semiconductor devices comprise bipolar transistors.

4. The external defibrillator of claim 3 wherein the semiconductor devices comprise high voltage insulated gate bipolar transistors (IGBTs).

5. The external defibrillator of claim 4 wherein the IGBTs have a voltage rating in excess of 2000 volts.

6. The external defibrillator of claim 1 further comprising a control circuit responsive to the processor,
wherein the semiconductor devices include control electrodes;
wherein the control circuit is coupled to the control electrodes of the semiconductor devices; and
wherein the control circuit is operable to switch the semiconductor devices to a nonconducting state.

7. The external defibrillator of claim 6 wherein the semiconductor devices comprise IGBTs; and wherein the control electrodes comprise the gate electrodes of the IGBTs.

8. The external defibrillator of claim 1 wherein the semiconductor devices are mounted on a printed circuit board.

9. The external defibrillator of claim 8 wherein the printed circuit board and semiconductor devices are encased in a dielectric material.

10. The external defibrillator of claim 8 further comprising a defibrillator circuit board on which the processor is mounted;
wherein the encased printed circuit board and semiconductor devices comprise a high voltage module;
wherein the high voltage module is mounted on the defibrillator circuit board.

11. The external defibrillator of claim 10 wherein at least one component of the charging circuit is located on the defibrillator circuit board.

12. The external defibrillator of claim 11 wherein the component of the charging circuit which is located on the defibrillator circuit board is a transformer.

13. The external defibrillator of claim 12 wherein the transformer is a planar transformer.

14. The external defibrillator of claim 10 wherein the high voltage module further comprises a plurality of pins electrically connected to the high voltage printed circuit board for electrically connecting the high voltage module to the defibrillator circuit board.

15. The external defibrillator of claim 1, wherein the bridge circuit comprises a half bridge circuit.

16. The external defibrillator of claim 15, wherein the half bridge circuit includes
first and second semiconductor devices coupled to a patient electrode; and
first and second capacitors coupled in parallel with the semiconductor devices.

17. The external defibrillator of claim 16 wherein the semiconductor devices comprise bipolar transistors.

18. The external defibrillator of claim 17 wherein the semiconductor devices comprise high voltage insulated gate bipolar transistors (IGBTs).
